(19) United States
(12) Patent Application Publication
(10) Pub. No.: US 2026/0224858 A1
(43) Pub. Date: Aug. 6, 2026

(54) OPHTHALMIC ARTERY GUIDEWIRE FOR TREATMENT OF OPHTHALMIC DISEASES

(71) Applicant: J.D. Franco & Co., LLC, Plano, TX (US)

(72) Inventors: Michael CALHOUN, Lighthouse Point, FL (US); Zachary TEGELS, Minneapolis, MN (US); Jeffrey FRANCO, Plano, TX (US)

(73) Assignee: J.D. Franco & Co., LLC, Plano, TX (US)

(21) Appl. No.: 19/629,752

(22) Filed: Mar. 26, 2026

Related U.S. Application Data
(63) Continuation of application No. PCT/US2024/048777, filed on Sep. 27, 2024.
(60) Provisional application No. 63/586,864, filed on Sep. 29, 2023.

(51) Int. Cl. A61M 25/09; A61M 25/00; A61M 25/01

(52) U.S. Cl. CPC A61M 25/09; A61M 25/0108; A61M 2025/0008; A61M 2210/0612

(57) ABSTRACT

A neuro guidewire (nGW) device for treatment of obstructions within vasculature behind an eye of a subject may include a corewire having a plurality of tapered portions, including: a first portion at a distal end of the corewire, and having a first diameter, a second portion, proximal to the first portion, and having a second diameter that is greater than the first diameter, and a third portion, proximal to the second portion, the third portion having a third diameter that is greater than the second diameter. The nGW device may also include a coil provided around one or both of the first portion and the second portion.

### CROSS-REFERENCE TO RELATED APPLICATIONS

This application is a continuation application of and claims the benefit of priority to PCT Application No. PCT/US2024/048777, filed on Sep. 27, 2024, which claims the benefit of priority to U.S. Provisional Patent Application No. 63/586,864, filed on Sep. 29, 2023, which are both incorporated herein in their entireties.

### FIELD OF INVENTION

The present disclosure relates to intravascular guidewire devices and to methods of using the same. More specifically, the present disclosure relates to a device having a neuro guidewire (nGW) (or an ophthalmic artery guidewire) for use as part of a system for treatment of obstructions (e.g., stenosis, lesions, and plaques) within neurovascular anatomy for the purpose of treating ophthalmic diseases.

### BACKGROUND

Guidewires (GWs) are routinely used to diagnose and treat diseases in the peripheral, coronary, and neurovascular anatomy. The first GW procedure was conducted by Dr. Charles Dotter in 1964, and GWs continue to be an integral part of intravascular diagnostic and therapeutic procedures today. Conventional GW lengths range from about 135 cm to about 300 cm. GWs typically contain a solid corewire with a flexible distal coil or spring, often made from a radiopaque material. The corewire is tapered on the distal end to increase its flexibility. The coil spring is typically affixed to the corewire on the distal end by welding or soldering proximally at a point where the inside diameter of the coil spring matches the outside diameter of the corewire and distally at the distal tip. Platinum is usually selected as the material for the coil spring because it provides radiopacity for visualization under fluoroscopy during navigation of the GW in the body. The distal most tip of the GW may be designed to facilitate user shaping to improve the steerability. Steering the GW is accomplished by rotationally manipulating the proximal most section of the wire while advancing the GW under fluoroscopy. In the early 1980s, the first steerable GWs were designed for specific use in the neuro circulation (that is, in the neurovascular anatomy). These GWs were designed for intracranial navigation in conjunction with a microcatheter. The use of GWs in the neurovascular anatomy allowed for the development and use of devices to treat aneurysms, clots, and other neurological issues. Neuro guidewires (nGWs) continue to be an integral part of neuro interventional procedures today. However, manufacture and use of an nGW device for treatment of and within the ophthalmic artery (OA) has not been previously contemplated. Existing devices do not address the anatomy for this application.

The invention of the present disclosure is related to intravascular guidewire devices and to methods of using the same, and, more specifically, to an nGW device, including an nGW, for use as part of a system for treatment of obstructions (e.g., stenosis, lesions, plaques) within neurovascular anatomy for the purpose of treating eye diseases (that is, ophthalmic diseases). In one particular embodiment, one example of use of this nGW device is in a system for use in the ophthalmic artery (OA) for the treatment of age-related macular degeneration (AMD).

### SUMMARY

In one aspect, a neuro guidewire (nGW) device for treatment of obstructions within vasculature behind an eye of a subject may include a corewire having a plurality of tapered portions, including: a first portion at a distal end of the corewire, and having a first diameter; a second portion, proximal to the first portion, and having a second diameter that is greater than the first diameter, and a third portion, proximal to the second portion, the third portion having a third diameter that is greater than the second diameter, and a coil provided around one or both of the first portion and the second portion.

In another aspect, a method of treating obstructions within vasculature behind an eye of a subject uses a neuro guidewire (nGW) device, the nGW device comprising a corewire having a plurality of tapered portions, including a first portion at a distal end of the corewire, and having a first diameter, a second portion, proximal to the first portion, and having a second diameter that is greater than the first diameter, and a third portion, proximal to the second portion, the third portion having a third diameter that is greater than the second diameter, and the nGW device further comprising a coil provided around one or both of the first portion and the second portion. The method may include inserting the nGW device into an ophthalmic artery (OA) via an internal carotid artery (ICA), and advancing a distal tip of the nGW device to a target area within the OA.

In still another aspect, a method of treating a disease within vasculature behind an eye of a subject uses a neuro guidewire (nGW) device, the nGW device comprising a corewire having a plurality of tapered portions, including a first portion at a distal end of the corewire, and having a first diameter, a second portion, proximal to the first portion, and having a second diameter that is greater than the first diameter, and a third portion, proximal to the second portion, the third portion having a third diameter that is greater than the second diameter, and the nGW device further comprising a coil provided around one or both of the first portion and the second portion. The method may include inserting a catheter sheath into a subject, inserting a guidewire (GW) through the catheter sheath to an internal carotid artery (ICA) of the subject, inserting a guiding catheter over the GW to the ICA, inserting an intermediate catheter over the GW to the ICA, inserting the nGW device through the intermediate catheter, into an ophthalmic artery (OA) via an internal carotid artery, and advancing a distal tip of the nGW device to a target area within the OA.

### BRIEF DESCRIPTION OF THE DRAWINGS

FIG. 1 a digital image of vasculature behind an eye of a human subject, and in particular, shows an ophthalmic artery (OA) and an internal carotid artery (ICA).

FIG. 2A is a digital image depicting the vasculature of a subject without age-related macular degeneration (AMD), and FIG. 2B is a digital image depicting the vasculature of a subject with AMD.

FIG. 3A is a digital image showing histopathology of a normal OA as it branches from the ICA, FIG. 3B is a digital image showing histopathology of an OA blocked at the ostium of the OA and the ICA, and FIG. 3C is a digital image showing histopathology of medial calcifications in the short limb (SL) of the OA.

FIG. 4A is a digital image that shows an nGW device prolapsing as it is advanced into the OA, and FIG. 4B is a digital image that shows an nGW device successfully advanced through the OA and navigated down to and past the central retinal artery (CRA).

FIG. 5A is a digital image of an OA branching off of an ICA and FIG. 5B is a detail view of a takeoff angle θ, at the origin of the OA shown in FIG. 5B.

FIG. 6 is a digital image depicting a length from an origin of an OA to a CRA branch.

FIG. 7 is a diagram showing an ICA and an OA.

FIG. 8A, FIG. 8B, and FIG. 8C are digital images depicting three examples of OA takeoff angles.

FIG. 9 is a map of the Bouthillier classification system.

FIG. 10 is a flowchart of a method of using an nGW device, in accordance with the present disclosure.

FIG. 11 shows an nGW device according to one embodiment.

FIG. 12 depicts a first portion of a corewire of an nGW device, according to one or more embodiments, positioned in a SL of an OA.

FIG. 13 depicts the first portion of a corewire of an nGW device, according to one or more embodiments, positioned in angle ‘b’ of an OA.

FIG. 14 depicts a portion of a third portion of a corewire of an nGW device, according to one or more embodiments, positioned in an OA.

FIG. 15 shows an nGW device according to another embodiment.

FIG. 16 shows an nGW device according to still another embodiment.

FIG. 17 shows an nGW device according to yet another embodiment.

FIG. 18A, FIG. 18B, and FIG. 18C show an nGW device according to another embodiment.

### DETAILED DESCRIPTION

Various embodiments of the present disclosure relate generally to devices for use in intravascular procedures for treatment of obstructions within neurovascular anatomy, and related methods of using the same.

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass±10% of a specified amount or value. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more. As used herein, the terms “comprises,” “comprising,” “including,” “having,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” In addition, the term “between” used in describing ranges of values is intended to include the minimum and maximum values described herein. The term “proximal” is used to describe the end of a device that is located closest to an operator of the device when using a device on a subject, whereas the term “distal” is used to describe the end of a device that is located closest to a subject on whom the device is being used and farthest away from the operator.

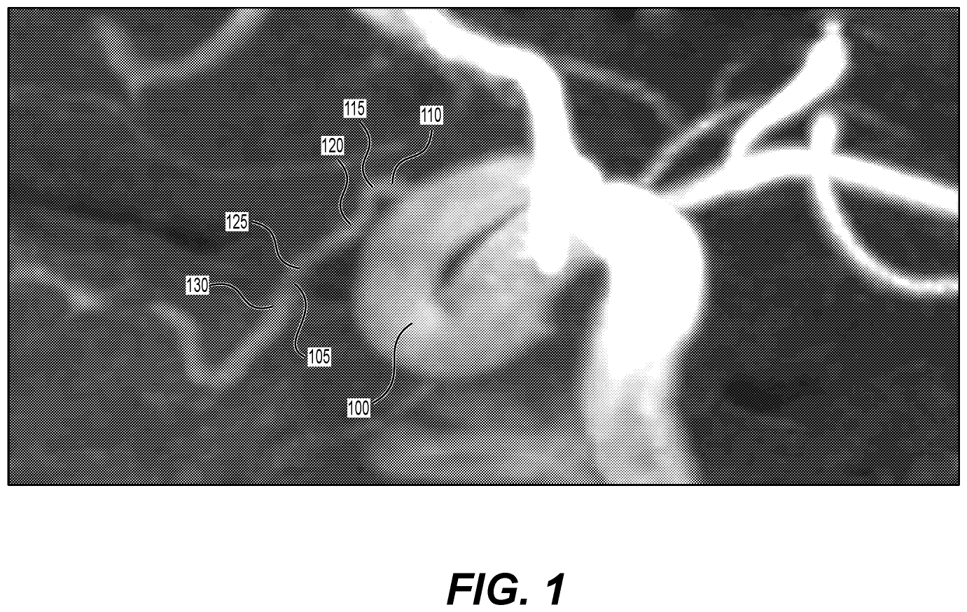

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. With reference to FIG. 1, anatomy of the vasculature behind an eye of a human subject will be described. In particular, FIG. 1 is a digital image showing the internal carotid artery (ICA) 100, and the ophthalmic artery (OA) 105, including the short limb (SL) 110 of the OA 105, an angle ‘a’ 115 of the OA 105, a long limb (LL) 120 of the OA 105, an angle ‘b’ 125 of the OA 105, and a distal part 130 of the OA 105. The OA 105 is an autoregulating, terminal branch of the ICA 100, and provides the majority supply of blood to the eye. FIG. 1 shows the OA 105 as a branch of the ICA 100, and depicts the arrangement of the OA 105, including the arrangement of the SL 110, the angle ‘a’ 115, the LL 120, the angle ‘b’ 125, and the distal part 130 of the OA 105, as it branches from the ICA 100 in a non-diseased vessel. The retina (not shown) is supplied with oxygenated blood by the OA 105, and rests in the back of the eye. It contains approximately 126 million photoreceptors. These photoreceptors consume more oxygen than any other tissue in the human body. Any disruption or blockage (i.e., interruption) of normal blood flow rates to the eye impacts the flow of nutrients into and the flow of waste products out of the eye. This flow alteration may cause a lack of oxygen flow (via a lack of blood flow) to the photoreceptors and allow the accumulation of waste products. These conditions may cause devastating injury to photoreceptors and in turn have a negative effect on vision. In some cases, irreversible vision damage may result.

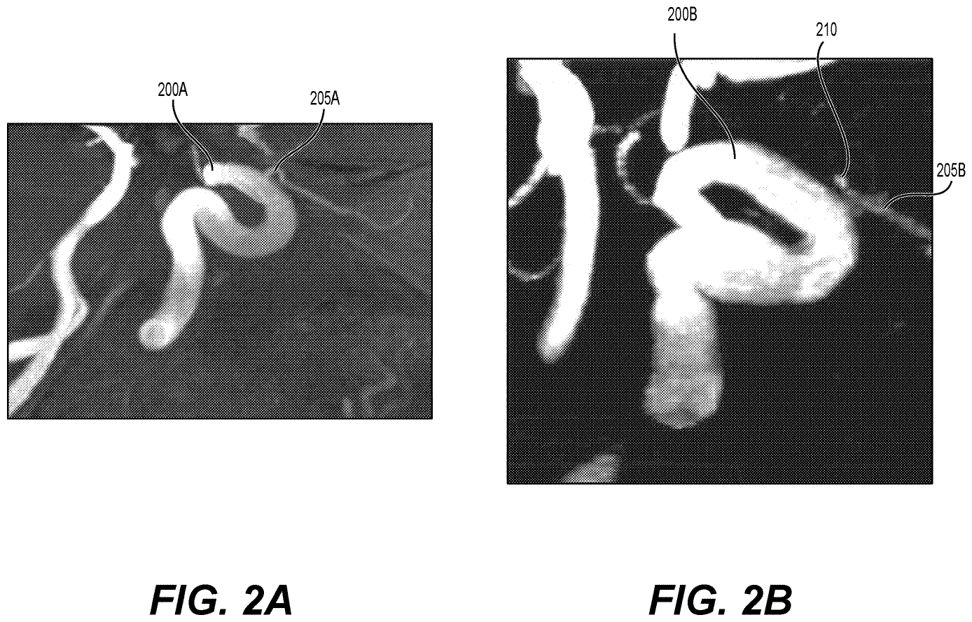

FIG. 2A is a digital image depicting the vasculature of a subject without age-related macular degeneration (AMD), including an ICA 200A and an OA 205A, and FIG. 2B is a digital image depicting the vasculature of a subject with AMD, with an ICA 200B and an OA 205B. Note the OA 205B of the subject with AMD, shown in FIG. 2B, is blocked by stenosis 210.

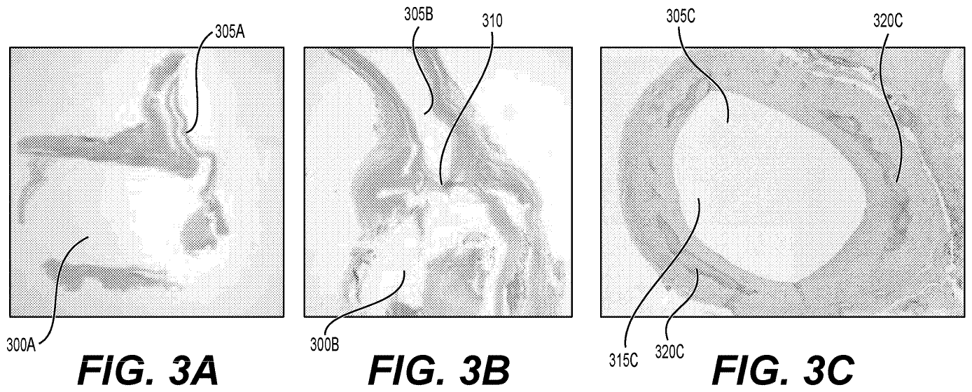

FIG. 3A is a digital image depicting histopathology of a normal OA 305A as it branches from an ICA 300A, FIG. 3B is a digital image depicting histopathology of an OA 305B that is blocked at the ostium of the OA 305B and an ICA 300B by a lesion 310 (also referred to as a total occlusion OA), and FIG. 3C is a digital image depicting histopathology of an SL 315C of an OA 305C, with medial calcifications 320C. The vasculature of FIGS. 3B and 3C would be treatment targets for the devices and methods of the present disclosure.

Treatment of the OA, such as OA 205B in FIG. 2B, the OA 305B in FIG. 3B, or the OA 305C in FIG. 3C, may be indicated in cases where luminal stenosis is 50% or less. In one example of a method of use of neuro guidewire (nGW) devices of the present disclosure, OA lesions in the range of 20% to 40% stenosis may be treated. It has not been previously contemplated that treatment of lesions with less than 50% stenosis would be meaningful. As noted above, the photoreceptors of the retina consume more oxygen than any other tissue in the human body and even a luminal stenosis of less than 50% can have a devastating effect on the photoreceptors.

Table 1 lists examples of OAs, and specifically, OAs of specific diameters, cross-sectional areas thereof, percentage decrease in cross-sectional areas, and percentage stenosis based on the percentage decrease in cross-sectional areas. In particular, in example OA number 2, a 16% stenosis has an impact equivalent to a 30% reduction in cross-sectional blood flow area. Further, in example OA number 4, a 50% stenosis has a 75% reduction in cross-sectional blood flow area. At least these two example OAs show that luminal stenosis of 50% or less can have an impact equivalent to a percentage to a percentage decrease in cross-sectional area of, for example, 30.56%, 55.56%, or 75%, and, therefore, a luminal stenosis of 50% or less can significantly affect blood supply to the photoreceptors.

TABLE 1

| | | Cross-Sectional | Percentage | |
|---|---|---|---|---|
| Example | OA Diameter | Area (CSA) | Decrease in | Percentage |
| OA # | (mm) | (mm²) | CSA (mm²) | Stenosis |
| 1 | 1.20 | 1.1310 | 0.00 | 0.00 |
| 2 | 1.00 | 0.7854 | 30.56 | 16.67 |
| 3 | 0.80 | 0.5027 | 55.56 | 33.33 |
| 4 | 0.60 | 0.2827 | 75.00 | 50.00 |
| 5 | 0.40 | 0.1257 | 88.89 | 66.66 |
| 6 | 0.20 | 0.0314 | 97.22 | 83.33 |
| 7 | 0.00 | 0.0000 | 100.00 | 100.00 |

In addition to not appreciating lower levels (percentages) of stenosis having significant effect on blood flow through small diameter arteries, there is also a lack of appreciation for the complexity of the OA anatomy and the degree of difficulty in accessing and treating the OA anatomy using conventional tools. The most challenging aspect of this treatment is accessing the desired treatment location within the OA. Current nGW devices are not designed for use in this area (that is, in the vasculature behind the eye), and do not address this unique anatomy, which is smaller in diameter, and which is angulated and significantly more tortuous, as compared to typical neuro vessels or cardiovascular vessels and, because of that tortuosity, current GW devices do not provide adequate support to establish purchase for a therapeutic device to enter the OA.

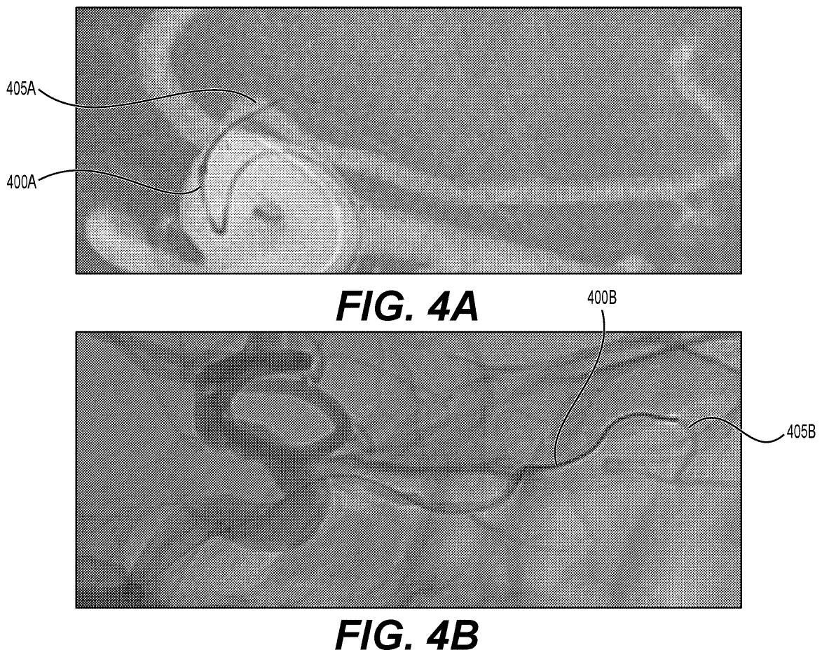

FIG. 4A is a digital image that shows an nGW device 400A prolapsing as it is advanced into an OA 405A, and FIG. 4B is a digital image that shows an nGW device 400B successfully advanced through the OA 405B and navigated down to the central retinal artery (CRA) (not shown). Placement of the nGW device 400B in the OA 405B down to and past the CRA allows the nGW device 400B to have proper purchase in the OA 405B so the physician can easily manipulate angioplasty balloons and other devices over the nGW device 400B to provide therapy or treatment.

In addition to placement down to the CRA, the nGW devices of the present disclosure are configured to access an ostium of an OA (that is, the location at which the OA meets the ICA) with an angle of takeoff, or the angle between the ICA and the OA branching therefrom) in the range of about 45° to about 140°, and to be placed within portions of the OA between the ostium down to the CRA.

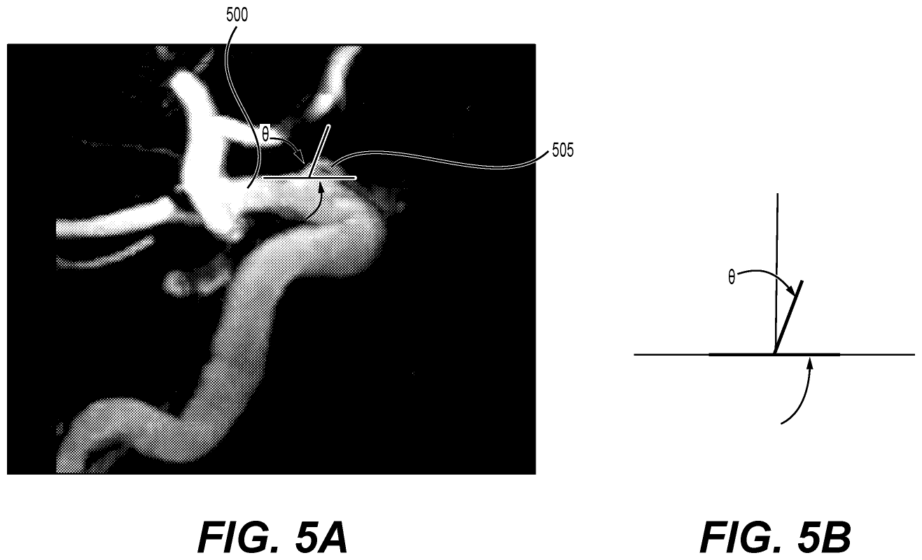

FIG. 5A is a digital image of an OA 505 branching off of an ICA 500, and, in particular, depicts a takeoff angle θ, at the origin of the OA 505, which contributes to the difficulty in accessing the OA 505, as the OA 505 branches from or takes off from the ICA 500. The takeoff angle θ, shown in detail in FIG. 5B, may be about 78°.

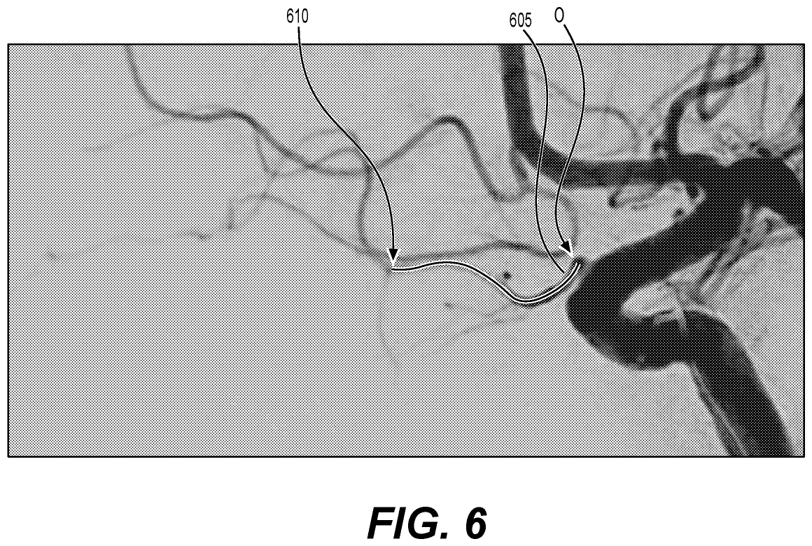

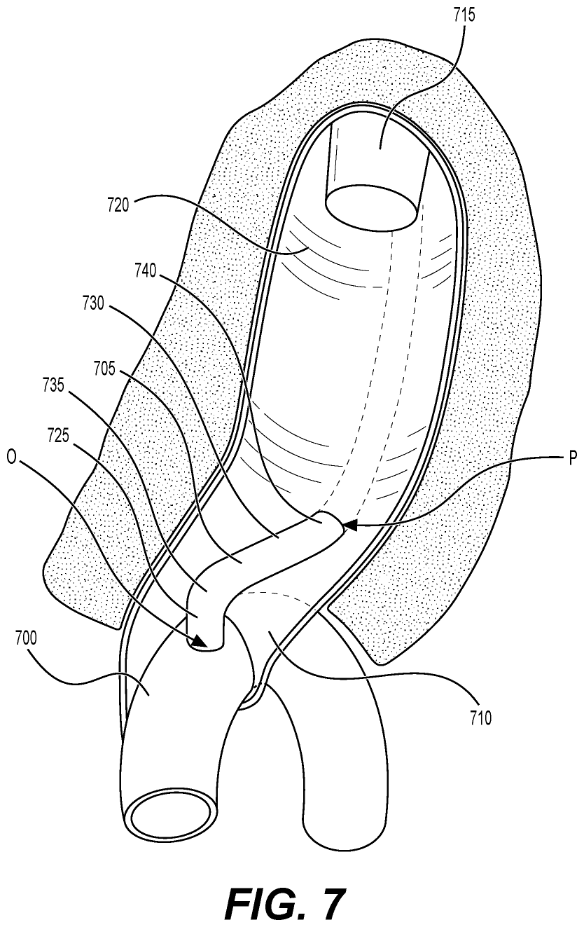

The nGW devices described herein are configured for access and placement of the nGW device within the OA, and to have optimized diametric section dimensions and overall lengths, with relatively improved flexibility, torquability, and pushability to address the unique anatomy of the vasculature behind the eye, including the OA. Optimization of these particular features and properties of the devices contributes to the ability of the nGW devices to access, properly locate or position, and aid in the delivery of therapy to the OA. The diameter of the OA as it branches off of the ICA, also referred to as the origin diameter, may range from about 1.2 mm to about 2 mm in a healthy human subject. Portions of the OA closer to the eye (in other words, more distal portions of the OA) typically have reduced diameters, which may be less than about 1 mm along the segment from the origin of the OA to the branch of the CRA. In the example of treatment of the OA, the target area includes the OA origin at the ICA and continues distally (that is, towards the eye), until just proximal to the branch of the CRA (that is, the CRA branch) from the OA. This total length may be in the range of about 10 mm to about 25 mm. FIG. 6 is a digital image depicting the length from the origin O of the OA 605 to the CRA branch 610, which may be approximately 21.4 mm. It may be desirable to treat anywhere along this portion or length of the OA 605 with the nGW devices and methods described herein.

An area of particular interest for treatment with an nGW device according to the present disclosure is the SL of the OA. As noted above, this is the initial branch of the OA off of the ICA. FIG. 7 is a diagram showing an ICA 700 and an OA 705. In particular, FIG. 7 shows an origin O of the OA 705 from the ICA 700, and more particularly, shows the ICA 700, a periosteum 710, a point P of penetration of a dural sheath (not shown), the OA 705, an optic nerve 715, an optic canal 720, an SL 725, a long limb 730 of the OA 705, angle ‘a’ 735 between the SL 725 and the LL 730, and angle ‘b’ 740 between the LL 730 and the portion of the OA 705 that is distal of the LL 730. Typical SL lengths that range from about 0.7 mm to about 2.7 mm.

As noted above, the OA may branch off of the ICA at an angle (that is, a takeoff angle) in a range of about 45° to about 140° and may contain partial or total occlusions and/or concentric or non-concentric calcified lesions. The takeoff angle and level of occlusion or calcification may contribute to difficulty and access of the OA. The takeoff angle challenge may be further complicated by the position of the OA on the segment of the siphon of the ICA. Different OA positions on the siphon contribute to an increase in access difficulty for an nGW device.

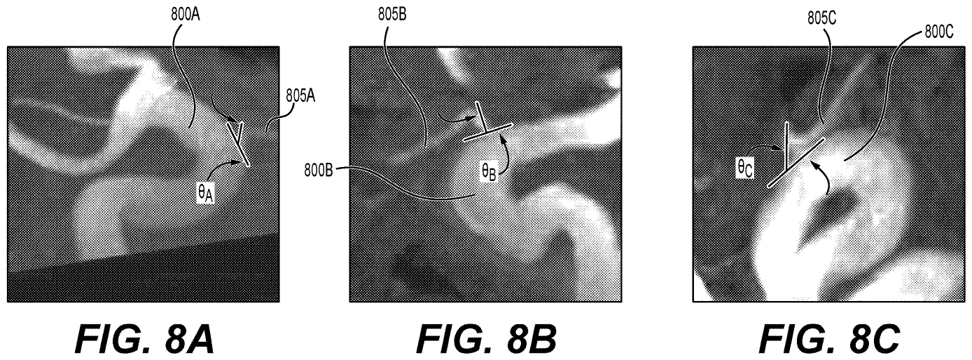

FIG. 8A, FIG. 8B, and FIG. 8C are digital images depicting three examples of OA takeoff angles ranging from 70° to 132°. Specifically, FIG. 8A depicts an OA 805A with a takeoff angle θ_{A} of 132° from the ICA 800A. FIG. 8B depicts an OA 805B with a takeoff angle θ_{B} of 74° from the ICA 800B. FIG. 8C depicts an OA 805C with a takeoff angle θ_{C} of 70° from the ICA. These images show OA origins located at different positions on the carotid siphon of the ICA.

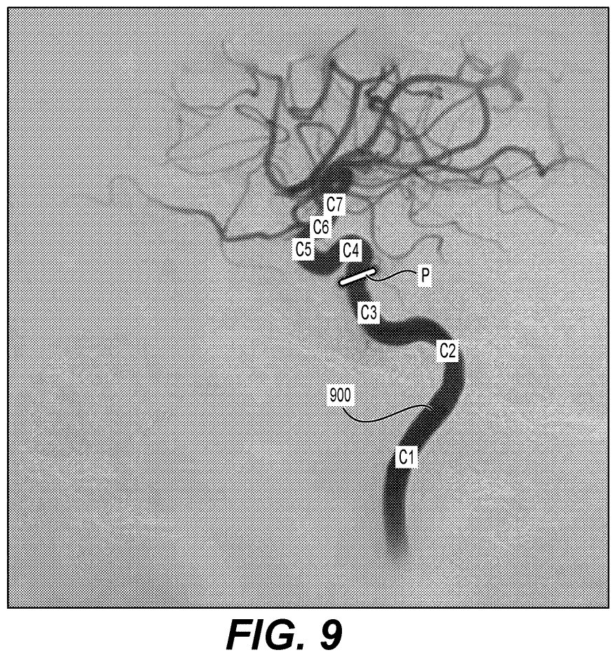

For the purposes of use of an nGW device in the OA, the Bouthillier classification system may be used to identify which ICA segment the nGW device would be positioned for treatment. FIG. 9 is a map of the Bouthillier classification system, and, in particular, shows segments of the carotid siphon of the ICA 900, including the target segments for use of an nGW device. FIG. 9 shows the cervical segment (C1 segment), the petrous segment (C2 segment), the lacerum segment (C3 segment), the cavernous segment (C4 segment), the clinoid segment (C5 segment), the ophthalmic segment (C6 segment), and the communicating segment (C7 segment). FIG. 9 also shows the petrolingual ligament P. In the case of the OA, a typical OA branch segment location is within one of the C5 segment or the C6 segment, also known as the clinoid or ophthalmic segments. As mentioned, this may vary according to the specific anatomy of the subject.

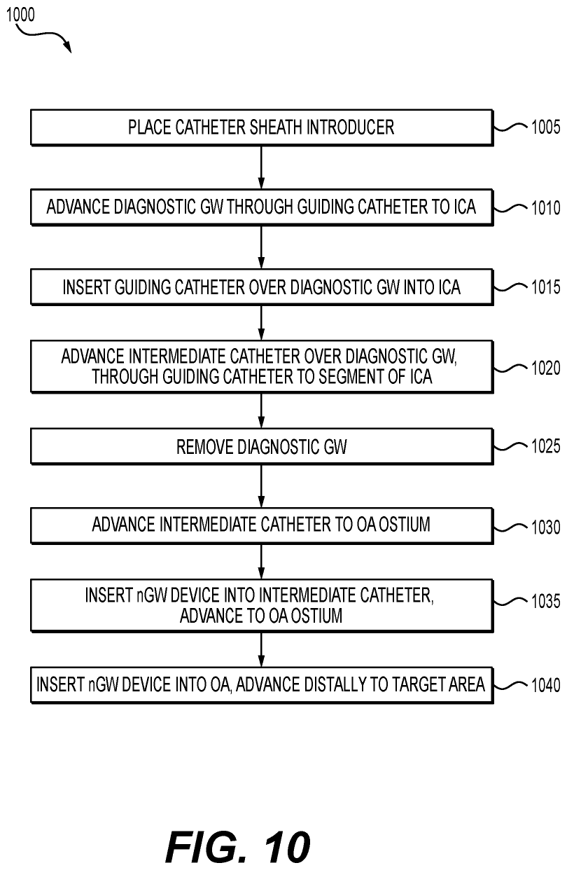

FIG. 10 is a flowchart of a method 1000 of using an nGW device within vasculature behind an eye of a subject for the treatment of a disease, such as AMD. Method 1000 may include a step 1005 of placing or inserting a catheter sheath, for example, an introducer, radially or femorally, into a subject. Then, in step 1010, a diagnostic GW may be inserted or advanced through the catheter sheath to the ICA. As an example, the diagnostic GW may be advanced to the C4 segment or the C5 segment of the ICA. The diagnostic GW may be, for example, 0.035 in (0.889 mm) or 0.038 in (0.965 mm) in diameter. Then, in step 1015, a guiding catheter of an appropriate size (i.e., diameter) may be advanced or inserted over the diagnostic GW, and navigated just past the common carotid artery bifurcation, into the ICA. Then, in step 1020, an intermediate catheter may be advanced or inserted over the diagnostic GW and through the guiding catheter to, for example, the C4 segment or the C5 segment. Then, in step 1025, the diagnostic GW may be removed, and, in step 1030, an intermediate catheter may be advanced as close as possible to the OA ostium. Then, in step 1035, the nGW device may be inserted into the intermediate catheter and navigated to the OA ostium to the C5 segment or the C6 segment. In step 1040, the nGW device may be inserted into the OA and advanced distally until the distal tip of the nGW device is in a target area or location within the OA. For example, the target location may be just proximal to the central retinal artery branch of the OA. The advancing of the nGW device may include using at least one of a marker (e.g., a marker band, including a radiopaque marker band) and a radiopaque coil, as well as using fluoroscopy, to track and confirm placement of a distal tip of the nGW device at the target area. Once the nGW device is in place, other devices (for example, an angioplasty balloon) may be advanced over the nGW device and placed at a target site (which may be the target area or location at which the distal tip of the nGW device is placed, or may be distal relative to the target area or location) so that a procedure or a treatment (for example, an angioplasty) may be completed. As one specific example, in a case in which an nGW device having a hypotube is used in the method 1000, the hypotube having one or more openings along its length and at least one marker provided adjacent to the one or more openings, the method 1000 may further include the steps of positioning the one or more openings of the hypotube at a second target area or location, and supplying a fluid to the second target area or location via the one or more openings of the hypotube. That is, a fluid may be supplied from a proximal end of the hypotube, and through the hypotube to the second target area. As noted above, use of the nGW device may be guided using fluoroscopy, for example.

Although the method 1000 is described as including steps 1005 to 1040, the method 1000 may include a subset of these steps and/or additional steps, as noted above. For example, the method 1000 may include the step 1040 of inserting the nGW device into the OA and advancing the nGW device distally until the distal tip is at the target area or location. The method 1000 may also include a step of molding a shaping segment in a first portion of a corewire of the nGW device into a desired shape prior to inserting and advancing the nGW device into the OA.

In one or more embodiments, an overall length of the nGW device may be in a range of about 180 cm to about 320 cm. The nGW device may include a corewire formed of a material that is metallic or a polymer, and which may be processed to insure there is no whipping of the corewire when torqued. Whipping occurs when torque is not effectively transmitted from one end of the corewire to the other end and may result in the corewire storing torque and, after a period of time, suddenly releasing the torque all at once. The corewire may have multiple tapers (that is, a plurality of tapered portions) located at or near a distal end. When combined with a coiled segment, the tapered portions may provide an nGW device having a variety in diameters, with the diameter increasing from the distal end towards a proximal section of the nGW device. These tapered portions are configured to optimize entry and transit of the nGW device into the OA, and, in particular, into the SL of the OA. A distal-most section of the corewire may have a shaping segment to allow for a placement of a coil over it and to facilitate shaping by either the manufacturer or the user. The coil may be fabricated of a radiopaque material, such as platinum, to produce a signature visible under fluoroscopy. The coil section may be fabricated as a single- or multi-filar wind to accommodate specific performance characteristics. In one or more embodiments, a marker, in the form of a marker band or a similar device, may be provided within the coil to provide for measuring capability. A distal-most coiled section of the nGW device may be coated with a polymer to improve mechanical performance. A portion of or the entire nGW device may be coated with a material, such as a hydrophilic coating, polytetrafluoroethylene (PTFE), or another other commonly used coating, to enhance lubricity.

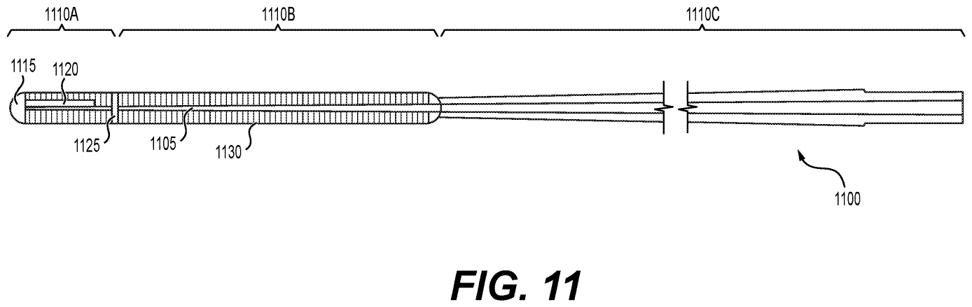

FIG. 11 shows an nGW device 1100 according to one embodiment. In this embodiment, the nGW device 1100 has a corewire 1105 with three tapered portions or segments, which may include a first, or distal-most, portion 1110A, at a distal end of the corewire 1105. The first portion 1110A may have a length of about 2 cm and a finished diameter of about 0.010 inch (in) (about 0.254 mm). The tapered portions may also include a second portion 1110B, proximal to the first portion 1110A. The second portion 1110B has a diameter that is greater than the diameter of the first portion 1110A. The second portion 1110B may have a length of about 5 cm and a finished diameter of about 0.012 in (about 0.305 mm). The first portion 1110A includes a distal tip 1115 of the nGW device 1100, and may also include one or both of a shaping segment 1120 proximal to the distal tip 1115 and a marker 1125 (e.g., a marker band). One or both of the first portion 1110A and the second portion 1110B may be covered with a coil 1130 (that is, the coil 1130 may be provided around one or both of the first portion 1110A and the second portion 1110B). The coil 1130 may be a radiopaque coil and/or may be coated with a material, such as a polymer material to improve lubricity. Specifically, the coil 1130 may be provided around the first portion 1110A and the second portion 1110B. A length of the coil 1130 may be about 7 cm, for example. The tapered portions may also include a third, or a final, portion 1110C. The third portion 1110C may have a diameter that is greater than a diameter of the second portion 1110B. Further, the third portion 1110C may make up the remainder of the overall length of the nGW device 1100, and may have a finished diameter of about 0.014 in (about 0.356 mm). The final length (or overall length) of the nGW device 1100 may be about 180 cm and/or the nGW device 1100 may have an exchange length of about 320 cm. The first portion 1110A may be configured to be shaped so that it will be easily manipulated to enter and transit the SL, which may have a length of about 0.7 mm to about 2.7 mm, up past angle ‘a’ of the OA.

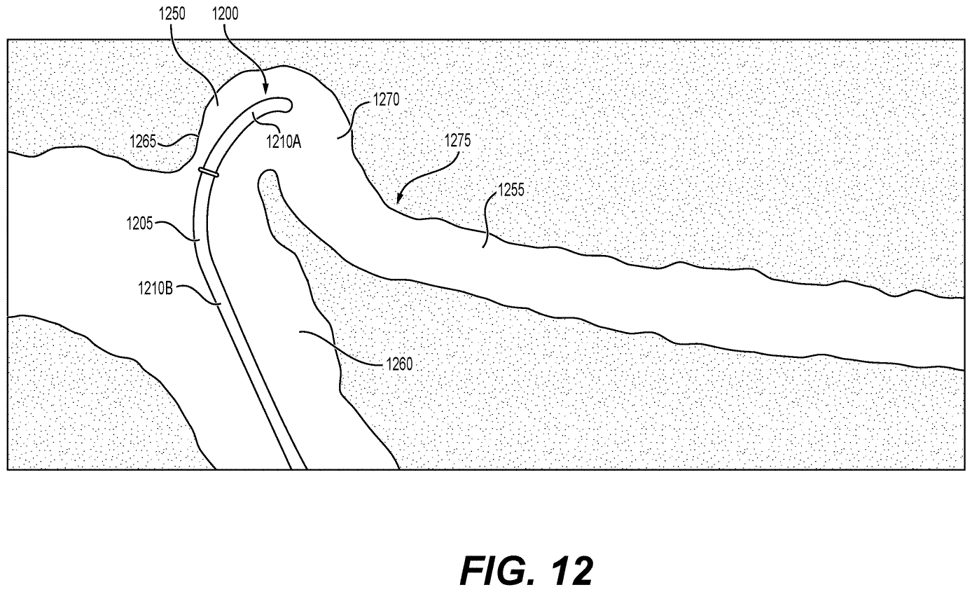

FIG. 12 depicts an nGW device 1200, which includes a corewire 1205 having at least a first portion 1210A, at a distal end of the corewire 1205, and a second portion 1210B proximal to the first portion 1210A. The nGW device 1200 may be, for example, the nGW device 1100 shown in FIG. 11. FIG. 12 shows the first portion 1210A positioned in the SL 1250 of the OA 1255 and the second portion 1210B positioned in the ICA 1260. Once the first portion 1210A is advanced past angle ‘a’ 1265, and enters the SL 1250 of the OA 1255, as shown in FIG. 12, the second portion 1210B may provide enough support to advance the first portion 1210B further into the OA 1255, past the LL 1270 and down to angle ‘b’ 1275.

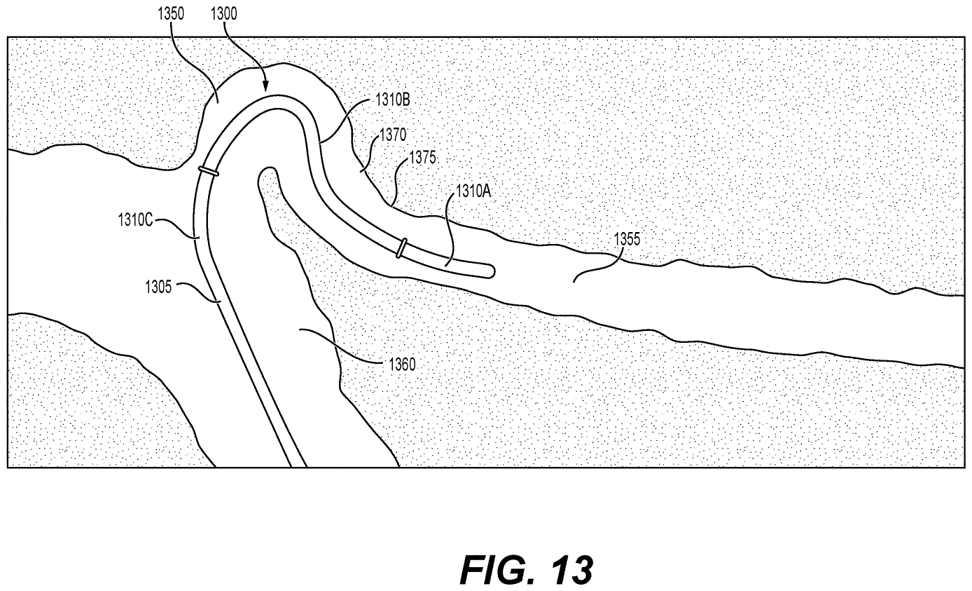

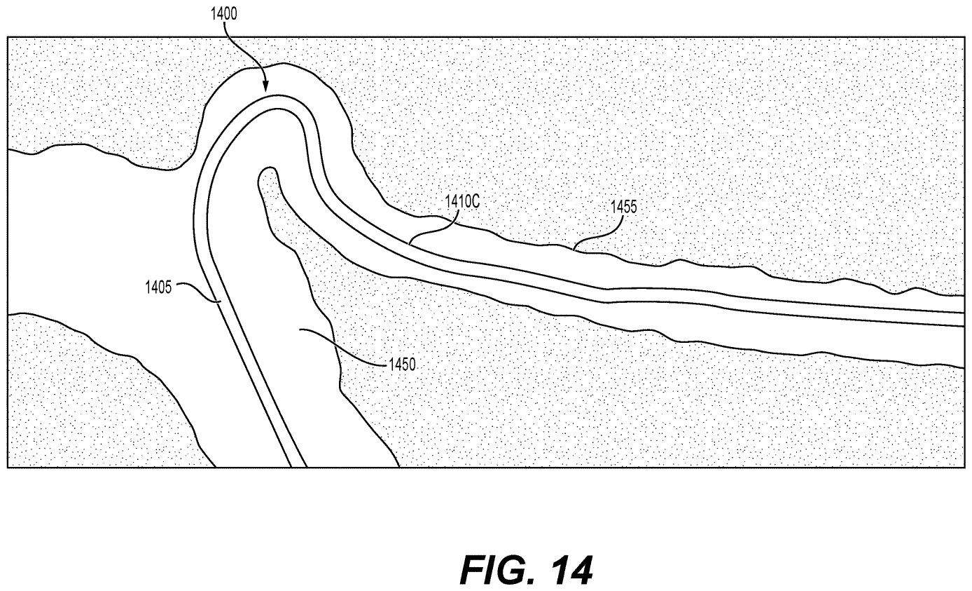

FIG. 13 depicts an nGW device 1300, which includes a corewire 1305 having a first portion 1310A, a second portion 1310B, and a third portion 1310C. The nGW device 1300 may be, for example, the nGW device 1100 shown in FIG. 11. FIG. 13 shows the first portion 1310A positioned in angle ‘b’ 1375 of an OA 1355, the second portion 1310B positioned in the SL 1350 and in the LL 1370, and the third portion 1310C positioned in the ICA 1360. Continuing description of the method of treatment referenced above with respect to FIG. 12, in FIG. 13, the nGW device 1300 is advanced so that the first portion 1310A has moved past the LL 1370 and down to angle ‘b’ 1375 of the OA 1355. The distance to move past the LL 1370 and down to angle ‘b’ 1375 may be in the range of about 1.4 mm to about 5.1 mm.

FIG. 14 depicts an nGW device 1400, which includes a corewire 1405 having a first portion 1410A (not shown), a second portion 1410B (not shown), and a third portion 1410C. The nGW device 1400 may be, for example, the nGW device 1100 shown in FIG. 11. FIG. 14 shows the third portion 1410C positioned in an OA 1455. Continuing description of the method of treatment referenced above with respect to FIG. 12 and FIG. 13, FIG. 14 shows the nGW device 1400 after it has been advanced so that at least a portion of the third portion 1410C, enters and advances within the OA 1455, without prolapsing of the nGW device 1400 into the ICA 1450. More specifically, the nGW device 1400 may be advanced until the third portion 1410C reaches a distance of approximately 11 mm into the OA 1455. This distance corresponds with a position that places a distal tip (not shown) of the nGW device 1400 near the CRA branch (shown in FIG. 6). This positioning of the nGW device 1400 allows for adequate purchase of the nGW device 1400 so that other coaxial devices may be advanced over the nGW device 1400 into the OA 1455 and reduces the chance of loss of position of the nGW device 1400 within the OA 1455.

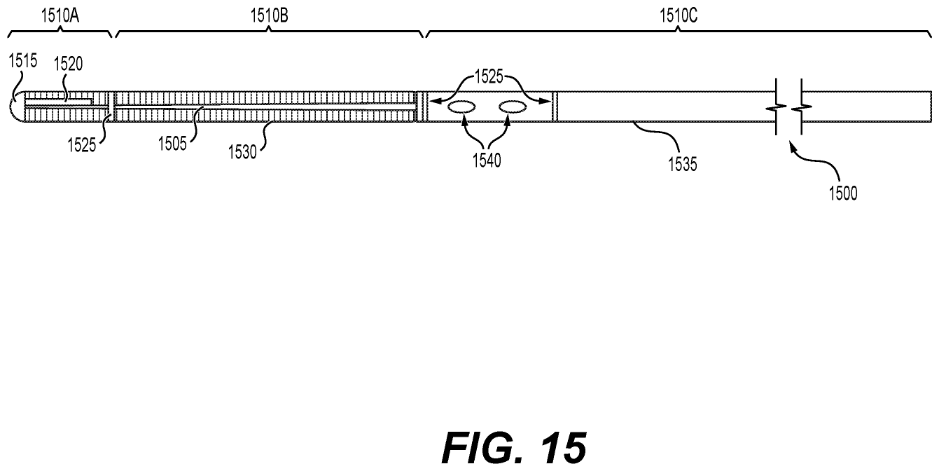

FIG. 15 shows an nGW device 1500 according to another embodiment. In particular, FIG. 15 shows the nGW device 1500 including a corewire 1505 having at least two tapered portions or segments, namely, a first, or distal-most, portion 1510A at a distal end of the corewire 1505. The first portion 1510A may have a length of about 2 cm and a finished diameter of about 0.010 in (0.254 mm). The tapered portions may also include a second portion 1510B, proximal to the first portion 1510A, with a diameter that is greater than the diameter of the first portion 1510A. The second portion 1510B may have a length of about 5 cm and a finished diameter of about 0.012 in (about 0.305 mm). In addition, the tapered portions may include a third portion 1510C, which may make up a remainder of the overall length of the nGW device 1500. A diameter of the third portion 1510C may be greater than a diameter of the second portion 1510B. The first portion 1510A includes a distal tip 1515 of the nGW device 1500, and may also include one or both of a shaping segment 1520 and a marker 1525 (e.g., a marker band), proximal to the distal tip 1515. One or both of the first portion 1510A and the second portion 1510B may be covered with a coil 1530. The coil 1530 may be a radiopaque coil, and/or the coil 1530 may be coated with a material, such as a polymer material to improve lubricity. Specifically, the coil 1530 may be provided around the first portion 1510A and the second portion 1510B. A length of the coil 1530 may be about 7 cm, for example. The nGW device 1500 of this embodiment may have a hypotube 1535, provided around the third portion 1510C. The hypotube 1535 may be fabricated of a metal or a polymer, either of which exhibits similar performance characteristics as a solid corewire design. The nGW device 1500 of this embodiment may provide for the ability to inject fluid through a distal portion of the nGW device 1500. For example, the nGW device 1500 may also include one or more holes or openings 1540 within the third portion 1510C, and through which fluid is supplied to vasculature from the nGW device 1500. In addition, one or more additional markers 1525 (e.g., marker bands) may be provided along the nGW device. In the example embodiment shown in FIG. 15, the nGW device 1500 may have two markers 1525 within the hypotube 1535. The markers 1525 may be provided around the openings 1540, to aid in positioning the openings 1540 and confirming placement before supplying fluid. The markers 1525 may aid in positioning the nGW device 1500 within vasculature, using, for example, fluoroscopy.

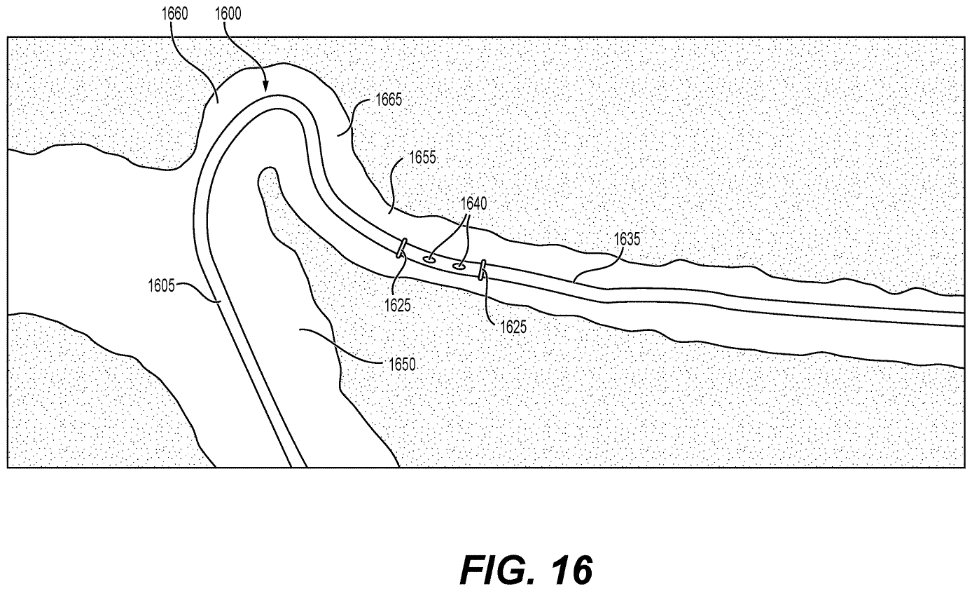

FIG. 16 shows an nGW device 1600 according to still another embodiment. The nGW device 1600 includes a corewire 1605 having at least a first portion (not shown), at a distal end of the corewire 1605, and a second portion (not shown), proximal to the first portion. The nGW device 1600 may be, for example, the nGW device 1500 shown in FIG. 15. FIG. 16 shows the hypotube 1635 positioned within the OA 1655, including a plurality of holes 1640. The nGW device 1600 is also shown with two markers 1625. In a method of use of such an nGW device 1600, the nGW device 1600 may be advanced into the ICA 1650 and the OA 1655, so that the distal portion, including the first portion (not shown), is at the CRA and the hypotube 1635 is within the SL 1660 and in a portion of the OA 1655 other than the SL 1660 (that is, a non-SL segment). When the hypotube 1635 is positioned within the LL 1665 of the OA 1655, a fluid may be injected into or supplied to the LL 1665 via the nGW device 1600, the fluid being supplied from the nGW device 1600 via the one or more holes 1640. In this embodiment, as an example, the fluid injected may include a calcium channel blocker or a contrast agent.

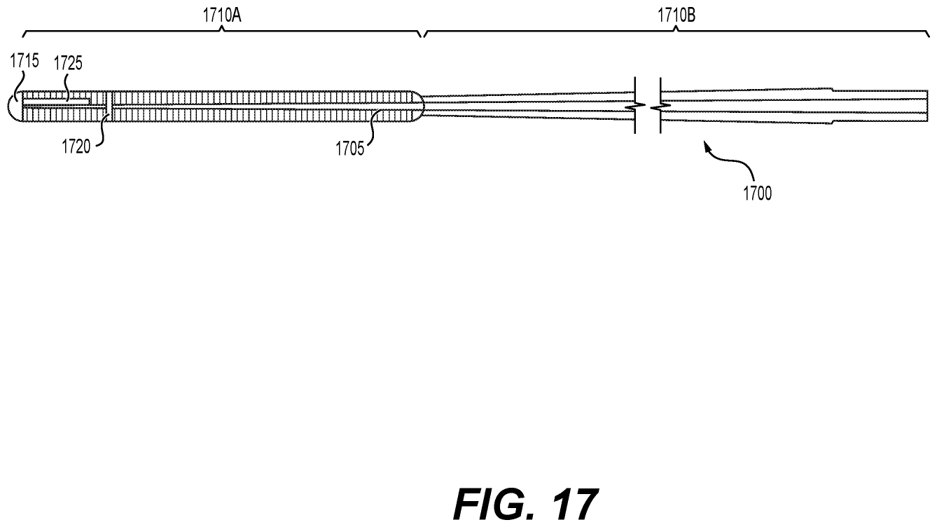

FIG. 17 shows an nGW device 1700 according to yet another embodiment. The nGW device 1700 of this embodiment includes a corewire 1705 having at least a first, distal-most portion 1710A, or a coil portion, and a second portion 1710B, proximal to the first portion 1710A. The first portion 1710A includes a distal tip 1715 of the nGW device 1700, and may also include one or both of a shaping segment 1720 and a marker 1725 (e.g., a marker band), proximal to the distal tip 1715. In addition, portions of one or both of the first portion 1710A or the second portion 1710B may be coated in a calcium channel blocker. More specifically, about 25 cm of the distal portion of the nGW device 1700 may be coated with a calcium channel blocker (e.g., nimodipine), which is typically used to treat vasospasm. Small diameter arteries may sometimes exhibit a condition called vasospasm which is when the vessel spontaneously constricts during an intravascular procedure and reduces the luminal diameter. This condition is typically treated by applying, injecting, introducing, or otherwise placing the calcium channel blocker into the affected artery. The calcium channel blocker stops the vasospasm and allows the artery to return to its normal size (that is, to a normal, non-reduced diameter). In this embodiment, the calcium channel blocker may be applied directly to a portion of the nGW device 1700 that is intended to be placed in an OA. The calcium channel blocker may be formulated to adhere to the nGW device 1700 but disperse in the blood stream as the nGW device 1700 sits in the OA. This disbursement of calcium channel blocker serves as a prophylactic measure, to prevent or reduce the occurrence of vasospasm. The coating of calcium channel blocker corresponds to what would be considered a therapeutic dose. The range of the dosage may depend on the length of the desired coating, and, as an example, may be in the range of about 0.25 mg to about 1.50 mg of delivered drug.

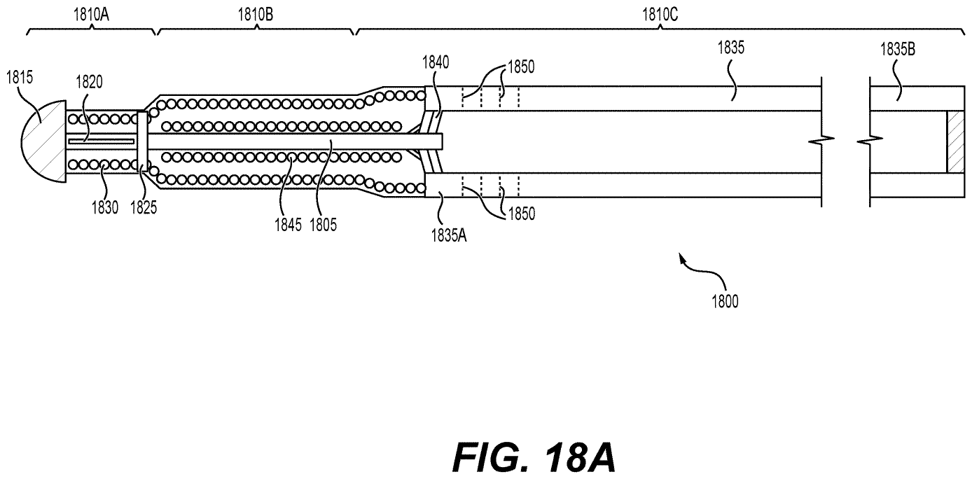

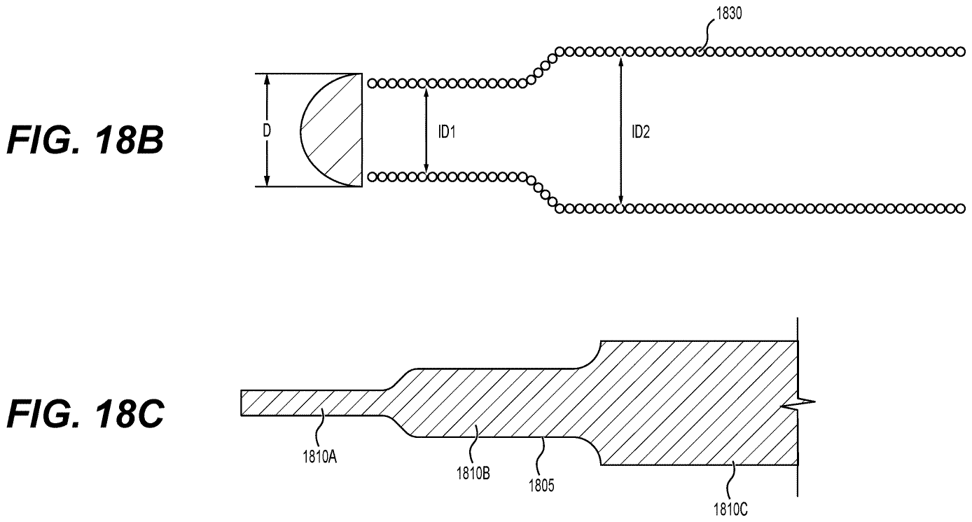

FIG. 18A, FIG. 18B, and FIG. 18C show an nGW device 1800 according to another embodiment. In this embodiment, the nGW device 1800 has a corewire 1805 having three tapered portions or segments which may include a first, or distal-most, portion 1810A. The first portion 1810A may have a length of between about 1 cm and about 3 cm and a finished diameter of about 0.010 in (about 0.254 mm). The tapered portions may also include a second portion 1810B, proximal to the first portion 1810. A diameter of the second portion 1810B may be greater than a diameter of the second portion 1810B. The second portion 1810B may have a length of between about 2 cm and about 6 cm, with a finished diameter of about 0.012 in (about 0.305 mm). The tapered portions may also include a third portion 1810C, proximal to the second portion 1810B. A diameter of the third portion 1810C may be greater than a diameter of the second portion 1810B. The third portion 1810C may have a length between about 1 cm and about 4 cm and finished diameter of about 0.014 in (about 0.356 mm). The third portion 1810C makes up the remainder of the overall length of the nGW device 1800, and has a finished diameter of about 0.014 in (about 0.356 mm). The final length (or overall length) of the nGW device 1800 may be about 180 cm and/or the nGW device 1800 may have an exchange length of about 320 cm. The first portion 1810A includes a distal tip 1815 of the nGW device 1800, and may also include one or both of a shaping segment 1820 and a marker 1825 (e.g., a marker band), proximal to the distal tip 1815. The first portion 1810A may be configured to be shaped so that it can be easily manipulated to enter and transit an SL up past angle ‘a’ of an OA. One or more of the first portion 1810A, the second portion 1810B, and the third portion 1810C may be covered with a coil 1830. The coil 1830 may be a radiopaque coil, and/or the coil 1530 may be coated with a material, such as a polymer material to improve lubricity. Specifically, the coil 1830 may be provided around the first portion 1810A, the second portion 1810B, and the third portion 1810C, as shown in FIG. 18A. The coil 1830 a polymer jacket, and/or may be coated with a material, such as PTFE, or a hydrophobic or hydrophilic material, alone or in in combination, to improve performance (e.g., to improve lubricity of the nGW device 1800). FIG. 18B is a detail view of the distal tip 1815 and the coil 1830, and shows the inner diameters ID1 and ID2 of the coil 1830, as well as an outer diameter D of the distal tip 1815, which may be about 0.010 inch (about 0.254 mm). The inner diameters FIG. 18C is a detail view of the corewire 1805, and in particular, shows the first portion 1810A, which is sized to fit within inner diameter ID1 of the coil 1830, the second portion 1810B, which is sized to fit within inner diameter ID2 of the coil 1830, and a final diameter, which is about 0.014 in (about 0.356 mm).

The nGW device 1800 may have a hypotube 1835 surround at least a portion of the third portion 1810C. The corewire 1805 may be joined to a distal end 1835A of the hypotube 1835 at a joint 1840. The joint 1840 between the corewire 1805 and the distal end of the hypotube 1835 may be, for example, a welded joint to allow the corewire 1805 to function as a torque transmission element. The nGW device 1800 may also include a multi-filar coil 1845 placed on the corewire 1805 to improve torque transmission. A distal portion of the nGW device 1800, the multi-filar coil 1845, and the corewire 1805 may be singularly or collectively constructed of a radiopaque material. The hypotube 1835 may include one or more holes or channels 1850 at the distal end, to facilitate delivery of a fluid, which may include, for example, drugs, saline, contrast, or other materials. The proximal end 1835B of the hypotube 1835 may be constructed so as to facilitate connection of a valve, such as a Tuohy-Borst valve or a similar device, to facilitate delivery of the fluid through the hypotube 1835. As noted above, the overall length of the nGW device 1800 of this embodiment may be in the range of about 180 cm to about 320 cm. In a case in which a length of the nGW device 1800 is relatively shorter (e.g., about 180 cm), the nGW device 1800 may also be connected to an exchange device to increase the overall length so as to facilitate GW exchange.

The embodiments of the nGW devices and the related methods described herein may provide for access and treatment of the OA for eye disease, including angioplasty of the OA, using a combination of IVL with balloon angioplasty and GWs, which may improve visual acuity in patients, such as patients with AMD. The IVL devices and related methods may also provide for treatment of calcified lesions to improve angioplasty outcomes. Further, the IVL devices and related methods of this disclosure may minimize the potential for embolic events in the retinal circulation, as a result of the OA angioplasty.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

We claim:

1. A neuro guidewire (nGW) device for treatment of obstructions within vasculature behind an eye of a subject, the nGW device comprising:
a corewire having a plurality of tapered portions, including:
a first portion at a distal end of the corewire, and having a first diameter;
a second portion, proximal to the first portion, and having a second diameter that is greater than the first diameter; and
a third portion, proximal to the second portion, the third portion having a third diameter that is greater than the second diameter; and
a coil provided around one or both of the first portion and the second portion.

2. The nGW device of claim 1, further comprising a hypotube provided around at least a portion of the third portion of the corewire.

3. The nGW device of claim 2, wherein the hypotube has one or more openings along its length, the one or more openings being configured to supply a fluid from within the hypotube.

4. The nGW device of claim 3, further comprising a plurality of markers, including at least one marker provided within the first portion, and at least one marker provided within the hypotube.

5. The nGW device of claim 1, wherein the coil is a radiopaque coil.

6. The nGW device of claim 1, wherein the coil is coated with a polymer.

7. The nGW device of claim 1, further comprising a shaping segment provided within the first portion of the corewire.

8. The nGW device of claim 1, wherein the first diameter is about 0.254 mm, the second diameter is about 0.305 mm, and the third diameter is about 0.356 mm.

9. The nGW device of claim 1, wherein an overall length of the nGW device is in a range of about 180 cm to about 320 cm, a length of the first portion is about 2 cm, a length of the second portion is about 5 cm, and a length of the third portion makes up a remainder of the overall length of the nGW device.

10. A method of treating obstructions within vasculature behind an eye of a subject using a neuro guidewire (nGW) device, the nGW device comprising a corewire having a plurality of tapered portions, including a first portion at a distal end of the corewire, and having a first diameter, a second portion, proximal to the first portion, and having a second diameter that is greater than the first diameter, and a third portion, proximal to the second portion, the third portion having a third diameter that is greater than the second diameter, and the nGW device further comprising a coil provided around one or both of the first portion and the second portion, the method comprising:
inserting the nGW device into an ophthalmic artery (OA) via an internal carotid artery (ICA), and advancing a distal tip of the nGW device to a target area within the OA.

11. The method of claim 10, wherein the target area is a location just proximal to a central retinal artery (CRA) of the subject.

12. The method of claim 10, further comprising advancing a device over the nGW device to perform a procedure or treatment.

13. The method of claim 10, wherein the nGW device further comprises a hypotube provided around at least a portion of the third portion of the corewire, the hypotube having one or more openings along its length and at least one marker provided adjacent to the one or more openings.

14. The method of claim 13, further comprising:
positioning, using the at least one marker, the one or more openings of the hypotube at a second target area within the OA; and
supplying a fluid from within the hypotube to the second target area within the OA via the one or more openings in the hypotube.

15. The method of claim 10, wherein the coil is a radiopaque coil, and wherein advancing the nGW device to the target area may include tracking and confirming placement of a distal tip of the nGW device at the target area using at least the radiopaque coil and fluoroscopy.

16. The method of claim 10, wherein the coil is coated with a polymer.

17. The method of claim 10, wherein the nGW device further comprises a shaping segment provided within the first portion of the corewire, and the method further comprises molding the shaping segment into a desired shape prior to inserting and advancing the nGW device into the OA.

18. The method of claim 10, wherein the first diameter of the corewire of the nGW device is about 0.254 mm, the second diameter is about 0.305 mm, and the third diameter is about 0.356 mm.

19. The method of claim 10, wherein an overall length of the nGW device is in a range of about 180 cm to about 320 cm, a length of the first portion is about 2 cm, a length of the second portion is about 5 cm, and a length of the third portion makes up a remainder of the overall length of the nGW device.

20. A method of treating a disease within vasculature behind an eye of a subject using a neuro guidewire (nGW) device, the nGW device comprising a corewire having a plurality of tapered portions, including a first portion at a distal end of the corewire, and having a first diameter, a second portion, proximal to the first portion, and having a second diameter that is greater than the first diameter, and a third portion, proximal to the second portion, the third portion having a third diameter that is greater than the second diameter, and the nGW device further comprising a coil provided around one or both of the first portion and the second portion, the method comprising:
inserting a catheter sheath into a subject;
inserting a guidewire (GW) through the catheter sheath to an internal carotid artery (ICA) of the subject;
inserting a guiding catheter over the GW to the ICA;
inserting an intermediate catheter over the GW to the ICA;
inserting the nGW device through the intermediate catheter, into an ophthalmic artery (OA) via an internal carotid artery, and advancing a distal tip of the nGW device to a target area within the OA.
